(19) United States
(12) Patent Application Publication
(10) Pub. No.: US 2006/0216768 A1
(43) Pub. Date: Sep. 28, 2006

(54) Dioxetane-nanoparticle assemblies for energy transfer detection systems, methods of making the assemblies, and methods of using the assemblies in bioassays

(76) Inventors: Alison Sparks, N. Andover, MA (US); Zhixian Wang, Winchester, MA (US); Brooks Edwards, Cambridge, MA (US); Rouh-Rong Juo, Allston, MA (US)

Correspondence Address: MERCHANT & GOULD PC, P.O. BOX 2903, MINNEAPOLIS, MN, 55402-0903, US

(21) Appl. No.: 11/221,895

(22) Filed: Sep. 9, 2005

Related U.S. Application Data
(60) Provisional application No. 60/608,130, filed on Sep. 9, 2004.

(51) Int. Cl. G01N 33/533

(52) U.S. Cl. 435/7.92; 977/950; 977/900

(57) ABSTRACT

Assemblies comprising nanoparticles and chemiluminescent substrates such as dioxetanes are provided. The assemblies can be used in assays to detect the presence and/or amount of a single analyte or multiple analytes in a sample. Methods of making the assemblies are also described.

This application claims the benefit of Provisional U.S. Patent Application Ser. No. 60/608,130, filed Sep. 9, 2004, which is incorporated by reference herein in its entirety.

### BACKGROUND

1. Technical Field

The present application relates generally to nanoparticle assemblies and to assemblies comprising nanoparticles and chemiluminescent substrates.

2. Background of the Technology

Quantum dots possess unique photophysical properties. For example, quantum dots exhibit narrow {e.g., 30-50 nm full width at half maximum (fwhm)} and symmetrical emission spectra and can be excited with one excitation source. Quantum dots are disclosed in the following references: Ligler and Taitt, eds., “Optical Biosensor: Present and Future, Colloidal Semiconductor Quantum Dot Conjugates in Biosensing,”, pages 537-569 (2002); W. C. W. Chan and S. Nie, Science 1998, 281:2016-201 8; X. Gao and S. Nie, Trends in Biotech. 2003, 21:371-373; X. Wu et al., Nature Biotech. 2003, 21:4146; B. Dubertret et al., Science 2002, 298:1759-1762; D. Larson et al., Science 2003, 300:1434-1436; J. Jaiswal et al., Nature Biotech. 2003, 21:47-5 1; M. Akerman et al., Proc. Nat. Acad. Sci. 2002, 99:12617-12621.

Energy transfer to quantum dots has been reported. See, for example, van Orden et al., Nano Lett. 2001, 1:469-474; Mattoussi et al., Nature Mat. 2003, 2:630-638; Phys. Rev. B, 1996, 54:8633-8643; Phys. Rev. Lett. 2002, 89:186802-1 to 86801-4; Abstracts of Papers, 223^{rd} ACS National Meeting, April 2002, COLL-172; Nano Lett. 2002, 2:817-822. There are limitations, however, to the general use of quantum dots in energy transfer systems. See, for example, Mattoussi et al., Nature Mat. 2003, 2:630-638; Mattoussi et al., J. Am. Chem. Soc. 2004,126:301-310. For example, the size of quantum dots (e.g., 2-10 nm) typically falls in the range of Forster donor-acceptor distances required for efficient energy transfer (e.g., 2-6 nm). Therefore, acceptor or donor dyes must be closely associated with the surface of the quantum dot thereby limiting the usefulness of quantum dot energy transfer assemblies.

Dioxetanes have been used as chemiluminescent substrates for very sensitive detection of hydrolytic enzymes or enzyme-labeled biomolecules, such as DNA and RNA. Dioxetane substrates are disclosed in the following U.S. Pat. Nos. 4,931,223; 4,931,569; 4,978,614; 5,112,960; 5,145,772; 5,326,882; 5,330,900; 5,336,596; 5,543,295; 5,538,847; 5,547,836; RE 36,536; 5,582,980; 5,851,771; and 6,218,135 B1. These substrates typically emit in the blue region (i.e., ˜460 nm Eₘₐₓ). Dioxetane substrates having red-shifted emission maxima have also been reported. See, for example, U.S. Pat. Nos. 4,952,707 and 6,355,441; Edwards et al., J. Org. Chem. 1990, 55:6225; Matsumoto et al., Luminescence 1999,14:345-348; Matsumoto et al., Tetrahedron 1999, 55:631-6840; and Matsumoto et al., Tetrahedron Letters 1999, 40:4571-4574. A typical dioxetane emission bandwidth, however, can span 100 nm to 150 nm or more at fwhm. Thus, red tailing from the bluer emitting dioxetanes can overlap the emission spectra of red-shifted dioxetanes. These emission characteristics limit the use of dioxetanes in multi-channel systems. For example, narrow bandwidth filters may be required for resolution of the broad dioxetane emissions in a multi-channel system.

Dioxetane-based chemiluminescent detection methods that combine the instrumental simplicity and detection sensitivity of enzyme-activated dioxetane substrates with narrowed, easily resolvable emission bandwidths and red-shifted emission maxima would therefore be desirable, particularly in multi-channel systems.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is an illustration of an ionically associated dioxetane-nanoparticle energy transfer assembly comprising a nanoparticle having an anionically charged surface coated with a cationic chemiluminescent enhancing polymer and a negatively charged dioxetane substrate ionically associated with the cationic coating.

FIG. 2 is an illustration of an ionically associated dioxetane-nanoparticle energy transfer assembly comprising a nanoparticle having an anionically charged surface coated with a polyelectrolyte multilayer (i.e., PEM) coating produced by sequential assembly of oppositely charged organic molecules and a negatively charged dioxetane substrate is ionically associated with the outermost cationic layer of the coating.

FIG. 3 illustrates an ionically associated dioxetane-nanoparticle energy transfer assembly comprising a nanoparticle having a surface modified by covalently attaching alkylammonium groups thereto to generate an overall cationic surface charge and a negatively charged dioxetane substrate ionically associated with the cationic charges on the nanoparticle surface.

FIGS. 4A and 4B are graphs which illustrate energy transfer from chemically triggered dioxetanes to CdSe/ZnS nanoparticles wherein FIG. 4A shows chemiluminescent emission from the substrate (AMPPD-OAC) and FIG. 4B shows photoluminescent emission from the nanoparticle/substrate assembly.

FIG. 5 illustrates a covalently linked dioxetane-nanoparticle energy transfer assembly comprising a plurality of dioxetanes covalently linked to a surface of a nanoparticle, where the excited state donor is generated by enzymatic deprotection of the covalently linked dioxetane.

FIG. 6 shows the chemical structure of a dioxetane with an NHS linker arm that can be used for covalent attachment to a nanoparticle surface.

FIGS. 7A-7C illustrate various coupling strategies for covalently linking dioxetanes to nanoparticles.

FIG. 8 illustrates various linker dioxetanes comprising thiol or heteroarylamino groups that can be bonded to a nanoparticle surface via S—Zn or N—Zn bonding.

FIG. 9 is a schematic illustrating a sandwich immunoassay on a nanoparticle with chemiexcitation-inititated nanoparticle photoluminescence wherein an enzyme-labeled (e.g., alkaline phosphatase) detection antibody (96) deprotects a dioxetane enzyme substrate (97) to generate an excited state donor species (98) thereby initiating nanoparticle photoluminescence via non-radiative energy transfer from the donor to the nanoparticle.

FIG. 10A is a schematic illustrating the structure of a carboxylate-coated CdSe/ZnS nanoparticle.

FIGS. 10B and 10C are graphs showing emission intensity as a function of wavelength for fresh (FIG. 10A) and aged (i.e., four month old) (FIG. 10B) solutions of carboxylate-coated CdSe/ZnS nanoparticles in 0.1M AMP buffer at a pH of 9.5.

FIG. 11A is a schematic illustrating the structure of an ammonium-coated CdSe/ZnS nanoparticles.

FIG. 11B is a graph showing emission intensity as a function of wavelength for ammonium-coated CdSe/ZnS nanoparticles.

FIG. 12A is a graph of emissions intensity as a function of wavelength for a solution of carboxylate-coated CdSe/ZnS nanoparticles, Sapphire II and CDP-Star® chemiluminescent dioexetane substrate activated by alkaline phosphatase.

FIG. 12B is a graph of emissions intensity as a function of wavelength for a micture of carboxylate-coated CdSe/ZnS nanoparticles and Sapphire II activated by alkaline phosphatase.

FIG. 13A is a graph showing emission intensity as a function of wavelength for triethylammonium dioxetane in the absence of carboxylate-coated CdSe/ZnS nanoparticles.

FIG. 13B is a graph showing emission intensity as a function of wavelength for triethylammonium dioxetane in the presence of carboxylate-coated CdSe/ZnS nanoparticles.

### DETAILED DESCRIPTION

Definitions:

As set forth herein, a “nanoparticle” refers to a luminescent particle having a size of up to 1000 nm. The nanoparticle can have a size from about 1 nm to about 100 nm. The nanoparticle can have a size from about 1 rum to about 50 nm. By “size” or “nanoparticle size” we mean the size and/or shape of the particle, or the features of the particle, that determines the unique luminescent emission spectra. The nanoparticle can be nanocrystalline or amorphous in composition, and can comprise inorganic elements or inorganic compounds. Exemplary luminescent nanoparticles include, but are not limited to, semiconductor nanocrystals such as CdS, CdSe, CdTe, CdS/ZnS, CdSe/ZnS, CdTe/ZnS, nanoporous Si, Si nanoparticles, and doped metal oxide nanocrystals.

The design of dioxetane-nanoparticle assemblies that enable energy transfer (ET) from excited state dioxetane donor fragments to nanoparticle acceptors are described herein. These assemblies can be used in novel detection systems wherein the dioxetane operates as the source of excitation energy for generation of nanoparticle luminescence. Thus, when dioxetane fragmentation is initiated by hydrolytic enzyme or chemical activation, the potential energy of the four-membered peroxide ring is released to excite the aryl ester fragment to a singlet excited state. Relaxation of the singlet excited state fragment to ground state transfers energy to proximal nanoparticle acceptors, which then luminesce with emission governed by nanoparticle size.

According to some embodiments, the dioxetane-nanoparticle energy transfer assembly can use electrostatic charge attractions to bind donor and acceptor ET components together. For example, the nanoparticle anionic shell can be coated with polycationic enhancing polymers (e.g., BDMQ, Sapphire, Sapphire 11, TPQ, THQ, phosphonium polymers, and/or copolymers of ammonium and/or phosphonium monomers); then negatively charged dioxetane substrates (e.g., CDP-Star or TFE-CDP-Star) can associate with the polycationic enhancer coating for subsequent energy transfer to the nanoparticle acceptor upon dioxetane decomposition.

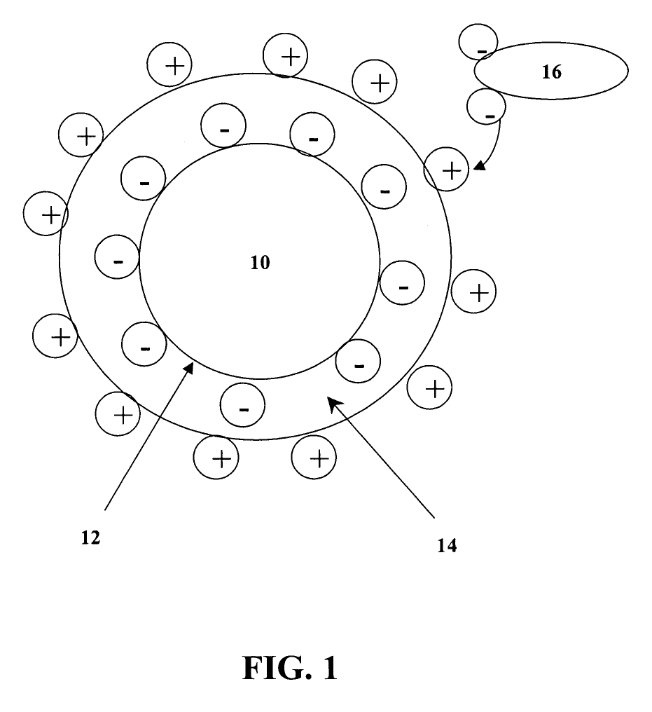

A chemiluminescent substrate/nanoparticle assembly comprising an ionically associated dioxetane substrate is shown in FIG. 1. As shown in FIG. 1, the outer shell of a nanoparticle (10) having an anionically charged surface (12) can be coated with a polycationic enhancer polymer to provide a particle with a positively charged surface (14). A negatively charged dioxetane (16) can then be ionically associated with the positively charged surface (14).

Alternatively, a polymeric enhancer-dioxetane hybrid, where the dioxetane is covalently attached to the enhancer polymer, can be ionically associated with the nanoparticle shell to coat the surface.

According to some embodiments, a charged nanoparticle surface can function as a support for a polyelectrolyte multilayer (PEM) coating, produced by sequential assembly of oppositely charged organic molecules. As with the ionic assembly example described above, the negatively charged dioxetane can associate with the outer cationic surface of a PEM-coated nanoparticle, and upon dioxetane decomposition, transfers excitation energy to generate nanoparticle luminescence. Polyelectrolyte multilayer coatings are disclosed in Decher, G. et al. eds., “Multilayer thin films: sequential assembly of nanocomposite materials”, Wiley-VCH, (2003); Harris, J. J. et al., J. Am. Chem. Soc., 1999, 121:1978-1979; and Locascio, L. E. et al., U.S. Patent Publication No. 2002-053514.

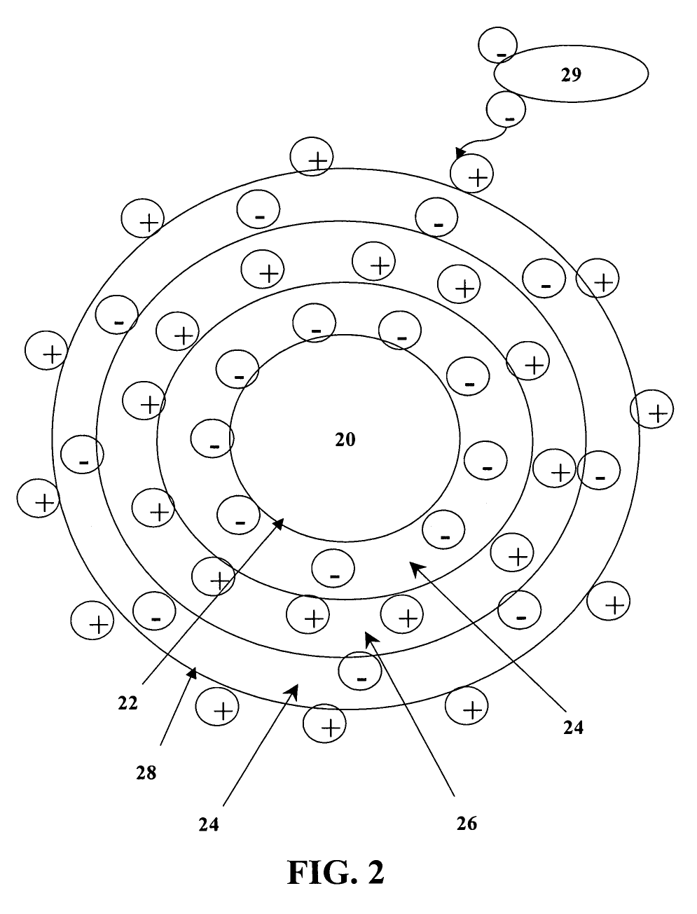

A chemiluminescent substrate/nanoparticle assembly comprising an ionically associated dioxetane substrate and a polyelectrolyte multi-layer (PEM) coating is shown in FIG. 2. As shown in FIG. 2, the outer shell of a nanoparticle (20) having an anionically charged surface (22) can be coated with a polyelectrolyte multi-layer (PEM) coating comprising alternating layers of a polycationic polymer (24) and a polyanionic polymer (26) to provide a particle with a positively charged surface (28). A negatively charged dioxetane (29) can then be ionically associated with the positively charged surface (28).

In some embodiments, the nanoparticle surface comprises covalently linked alkylammonium, phosphonium groups, or peralkylating surface amino groups that generate an overall cationic shell charge. The pH-independent peralkylammonium charge presents a surface of unvarying, strong cationic charge draw in negatively charged dioxetanes.

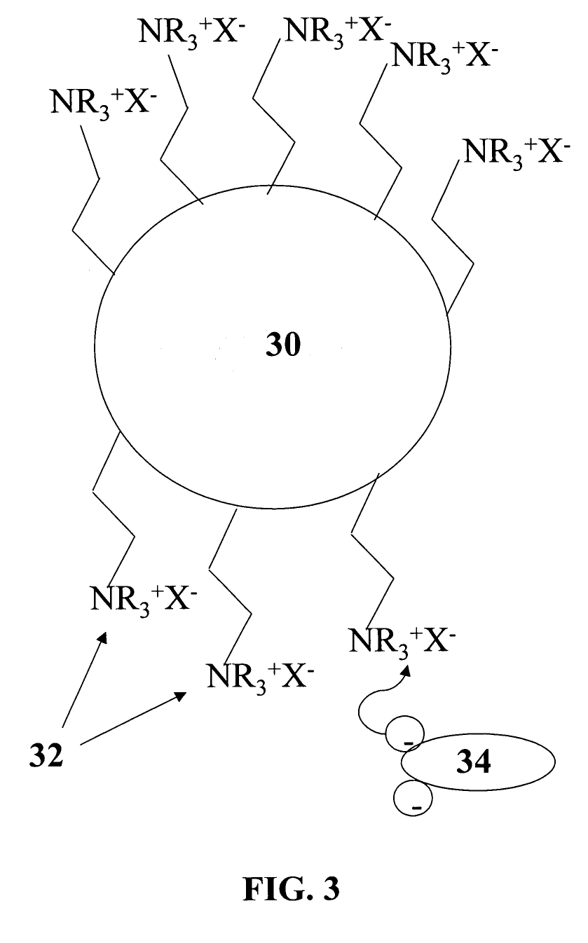

A chemiluminescent substrate/nanoparticle assembly comprising an ionically associated dioxetane substrate and a nanoparticle with covalently attached alkylammonium groups is shown in FIG. 3. As shown in FIG. 3, the outer shell of a nanoparticle (30) comprises a plurality of alkylammonium groups (32). A negatively charged dioxetane (34) can ionically associate with the positively charged alkylammonium groups (32).

A cationic nanoparticle shell in itself represents a novel invention, enabling other potential uses in an anion exchange mode. To our knowledge, all currently described nanoparticle surfaces are neutral or negatively charged.

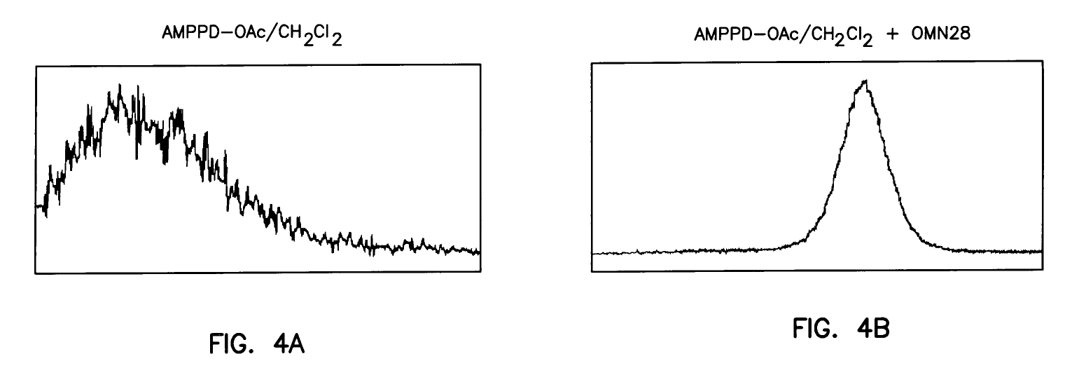

Energy transfer from chemically triggered dioxetanes to CdSe/ZnS nanoparticles has also been observed in a very specific organic solvent system. In this experiment, the AMPPD-OAC 460 nm emission (FIG. 4A) was quenched in the presence of CdSe/ZnS nanoparticles, resulting in red-shifted 555 nm emission (FIG. 4B).

According to a further embodiment, standard glass surface modification chemistry on the oxidized silicon shell of a silicon nanoparticle or nanoporous Si can be used to provide a convenient route to useful ionic nanoparticle surfaces to optimize formation and performance of ionically associated dioxetane-nanoparticle assemblies in a variety of biologically applications.

If the ionic associations are efficient enough to tightly hold an assembly of nanoparticle, polyionic enhancing layers, and dioxetanes, chemiexcitation of nanoparticle with dioxetanes provides a novel and potentially sensitive energy transfer detection method for aqueous-based analytes. The enhancing polymeric layers and/or PEMs can be coated onto the nanoparticle in the range of <1-50 nm in coating thickness. The penetration capability of the ionically associated chemiexcited species may vary depending on the nature and thickness of the ionic coating of the nanoparticle surface.

Although dioxetanes have been discussed above, in general, any ionically associated chemiluminescent moiety can be used as a chemiexcitation source for nanoparticle luminescence. Other examples of latent chemiluminescent donors include, but are not limited to, acridinium salt active esters, acridan esters or thioesters, acridan enol phosphates and other enol phosphates, and luminol substrates. Conversely, an anionic nanoparticle surface can be ionically associated with a cationically charged chemiluminescent substrate.

Nanoparticle beads are also provided wherein the acceptors comprise nanoparticle-embedded polymer beads with embedded nanoparticles that can expose a population of nanoparticles on the bead surface to chemiexcited species capable of energy transfer to the dots. These chemiluminescent substrate/nanoparticle ionic assemblies provide red-shifted luminescence initiated by chemiexcitation of chemiluminescent moieties such as dioxetanes. This excitation method reduces noise, does not require an external excitation light source, and provides luminescent signal red-shifted beyond typical autofluorescence bandwidths associated with biological and cellular materials. The chemiluminescent substrate/nanoparticle ionic assemblies can be used in multichannel detection systems with sequential detection or physically segregated detection channels or simultaneous detection in homogenous assay systems. The narrowed emission bandwidth of the nanoparticle acceptor enables easily resolvable luminescent signal in multichannel detection systems.

The dioxetane-nanoparticle ionic assemblies can be used as sensitive chemiluminescent detection substrates for enzyme labels in chip formats and biological assays in solution or on solid support. The ionically associated chemiluminescent substrate/nanoparticle assemblies can also be used to detect analytes bound to or captured by the surface of the nanoparticle or nanoparticle bead. For example, if an enzyme-labeled analyte is bound to the nanoparticle or bead surface, subsequent enzyme activation of the ionically associated dioxetane by the enzyme label can initiate chemiexcitation of nanoparticle luminescence.

According to some embodiments, a dioxetane molecule or multiple dioxetane molecules (e.g., 2-100 or more dioxetanes) can be covalently bound to the nanoparticle surface so that the distance between the energy donor (e.g., the excited dioxetane fragment) and the acceptor (nanoparticle) is minimized for optimal energy transfer.

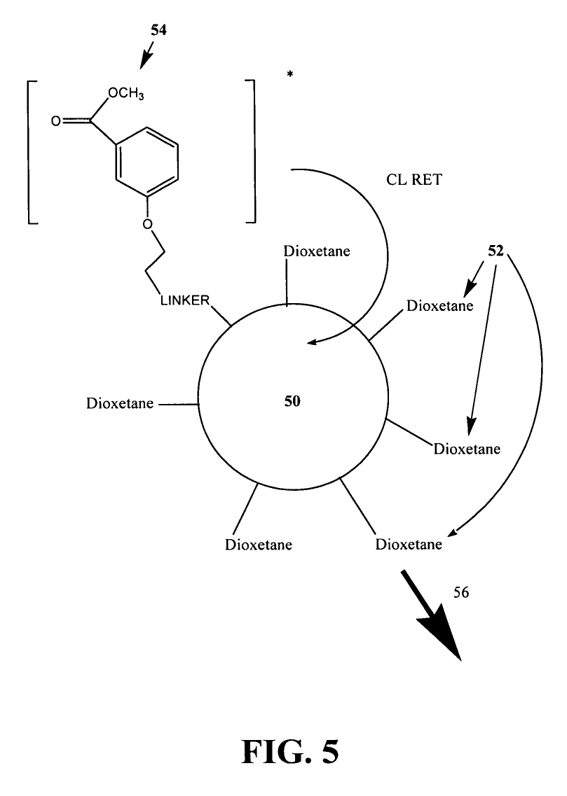

FIG. 5 shows a covalently linked dioxetane-nanoparticle assembly. As shown in FIG. 5, a plurality of dioxetanes (52) can be covalently linked to the surface of a nanoparticle (50). As also shown in FIG. 5, an excited dioxetane fragment (54) covalently linked to the nanoparticle surface can transfer energy to the nanoparticle resulting in photoluminescent emissions (56) from the nanoparticle. The nanoparticle emissions can have a wavelength of 480 nm to >700 nm.

According to some embodiments, a polymeric chemiluminescent enhancer and a chemiluminescent substrate (e.g., dioxetane) may both be covalently or noncovalently bound to the nanoparticle surface as separate entities, or the polymeric enhancer and dioxetane may be covalently bound to the nanoparticle shell as a hybrid polymeric enhancer-dioxetane structure, where the dioxetane is also covalently attached to the enhancer polymer. This particular energy transfer assembly design anchors the energy donor very near the nanoparticle surface so that large Forster energy transfer distances, that may be imposed by nanoparticle size, are held to a minimum.

The ability to covalently attach several to many dioxetanes results in multi-excitation of nanoparticle luminescence. If adapted to an enzyme-label detection system, enzyme amplification of signal by enzymatic turnover of chemiluminescent substrate, augmented by multi-excitation of a nanoparticle by multiple energy transfer events on a nanoparticle surface by multiple chemiexcited species, should give significantly amplified luminescent signal. The luminescent signal can be enhanced through efficient energy transfer to and emission from the nanoparticle alone, or the luminescent signal can be further enhanced by adding polycationic enhancer polymer to solutions, or by applying polycationic enhancer polymer to solid supports containing the covalent dioxetane-nanoparticle, or by applying polycationic enhancer polymer directly to the nanoparticle surface.

In general, any covalently linked chemiluminescent moiety can be used as a chemi-excitation source for nanoparticle luminescence. Other examples of latent chemiluminescent donors include, but are not limited to, acridinium salt active esters, acridan esters or thioesters, acridan enol phosphates and other enol phosphates, and luminol substrates. Nanoparticle beads can also be used where the acceptors are nanoparticle-embedded polymer beads that can expose a population of nanoparticles on the bead surface to chemiexcited species, generated from covalently bound chemiluminescent precursors, capable of energy transfer to the nanoparticle.

Standard glass surface modification chemistry on an oxidized silicon shell of a nanoparticle or on the surface of nanoporous Si or Si nanoparticles, can be used to provide funtional groups for covalently linking a variety of photochemically or biologically useful moieties to the nanoparticle.

Dioxetanes having chemically reactive functional groups that can be covalently linked to nanoparticle shell surfaces are also provided. For example, dioxetanes or the enol ether precursors, with linker arms end-modified with an NHS-ester can be coupled with amines, thiols, or alcohols on the nanoparticle surface, using standard literature conditions. Alternatively, dioxetanes or the enol ether precursors, with linker arms end-modified with a nucleophilic group such as amine, thiol or alcohol, can be coupled with moieties (e.g., activated esters or alkylhalides) present on the nanoparticle surface.

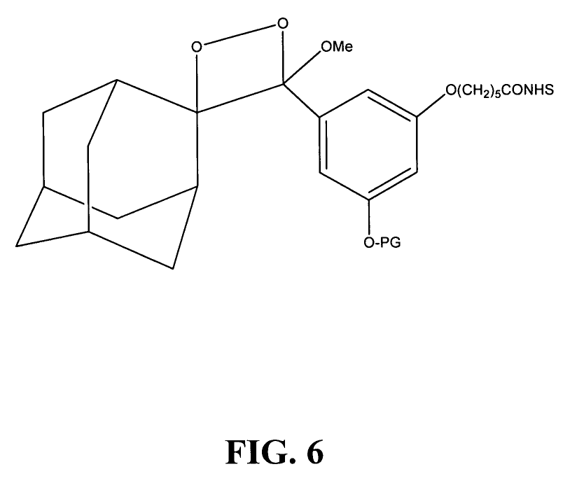

The chemical structure of an exemplary dioxetane comprising an NHS linker arm that can be used for covalent coupling to nanoparticle surfaces is shown in FIG. 6. In the structure shown in FIG. 6, the substituent “PG” represents a chemically or enzymatically cleavable protecting group.

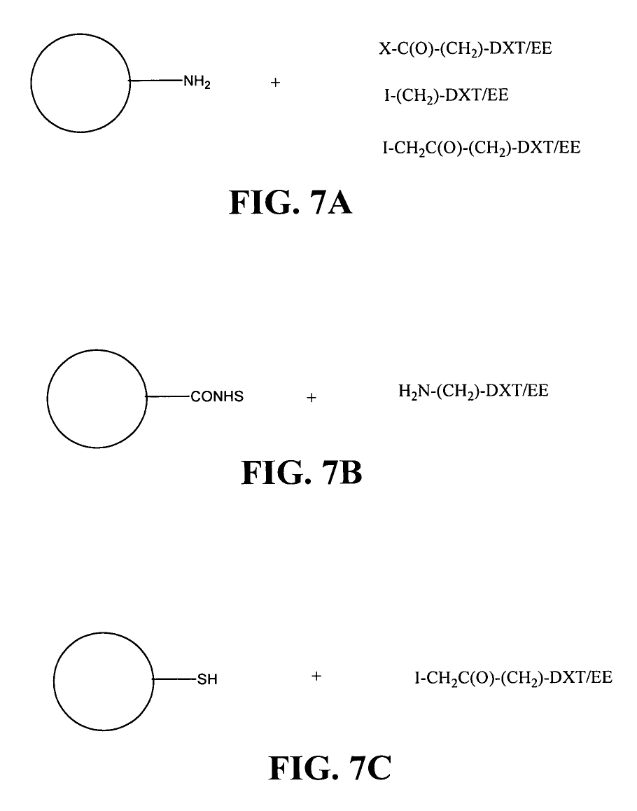

FIGS. 7A-7C illustrates various covalent coupling strategies for synthesizing covalently linked dioxetane-nanoparticle assemblies. FIG. 7A shows various strategies for covalent attachment of dioxetanes to nanoparticle surfaces comprising primary amine groups. FIG. 7B shows a strategy for covalent attachment of dioxetanes to nanoparticle surfaces comprising —C(═O)—N-hydroxy succinimidyl (CONHS) groups. FIG. 7C shows a strategy for covalent attachment of dioxetanes to nanoparticle surfaces comprising —SH groups. In FIGS. 7A-7C, “DXT” represents a dioxetane, “EE” represents an enol ether precursor, and “X” represents an activating group.

Standard glass surface modification chemistry can be used to provide functional groups for covalent attachment on surfaces of silicon-based nanoparticle. The use of silicon-based nanoparticles would avoid potential toxicity issues associated with Cd-based nanoparticles (See, for example, Bhatia et al., “Probing the Cytotoxicity of Semiconductor Quantum Dots”, Nano Letters 2004, 4:11-18).

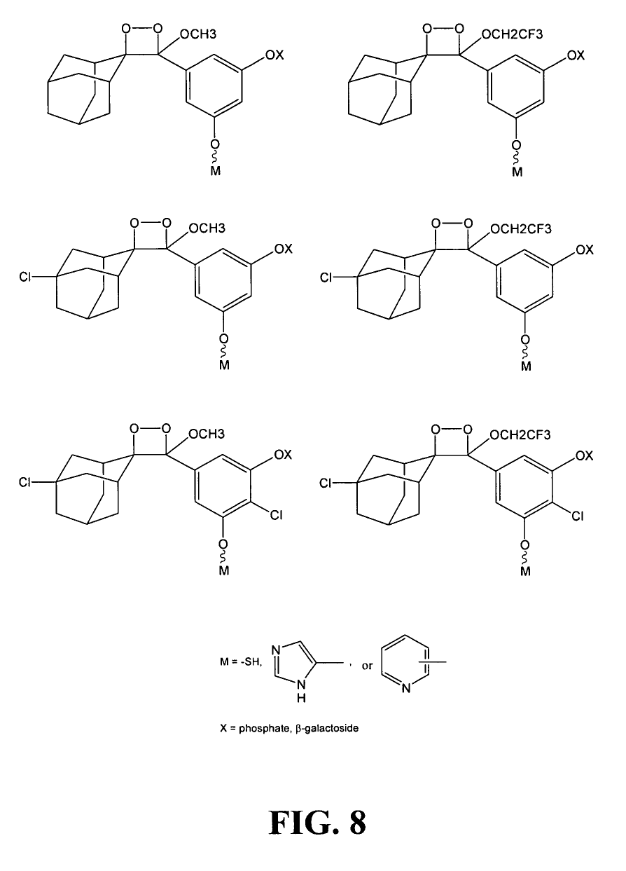

The chemical structures of exemplary dioxetanes comprising a linker arm that can be used for Zn—S or Zn—N bonding to the nanoparticle surfaces are shown in FIG. 8. In the structures shown in FIG. 8, the substituent “PG” represents a chemically or enzymatically cleavable protecting group. Bonding to the nanoparticle surface occurs by interaction of the surface with a linker group M that can be an alkylthio, arylthio, arylamino or heteroarylamino group, having a displaceable proton in the pKₐ range of 5.0-9.0.

The covalently linked dioxetane-nanoparticle assemblies can be used in multichannel detection systems with sequential detection or physically segregated detection channels. The covalently linked dioxetane-nanoparticle assemblies may also be used in simultaneous, multichannel detection systems. Homogenous mixtures of several discrete dioxetane-nanoparticle assemblies (e.g., assemblies A-D) can be used to simultaneously measure several analytes (e.g., analytes a-d). The narrowed emission bandwidth of the quantum dot acceptor enables easily resolvable luminescent signal in multichannel detection systems.

The covalently linked dioxetane-nanoparticle assemblies can be used as sensitive chemiluminescent detection substrates for enzyme labels in chip formats and biological assays in solution or on solid support. These energy transfer assemblies can also be used for simultaneous multichannel detection in homogeneous formats such as microarrays and immunoassays in solution or on solid support. The dioxetane-nanoparticle assemblies can also be used in intracellular assays.

Dioxetanes and nanoparticles appear to have low cytotoxicity (internal observation for dioxetanes; Dubertret et al., Science 2002, 298:1759-1762; Wu et al., Nature Biotech. 2003, 21:41-46; Gao and Nic, Trends in Biotech. 2003, 21:371-373; Akerman et al., Proc. Nat. Acad. Sci. 2002, 99:12617-1262 1; Mattoussi et al., Nature Biotech. 2003, 21:47-5 1; Webb et al., Science 2003, 300:1434-1436), and the capability of generating red-shifted, narrow emission bands allows luminescent signal detection outside the blue autofluorescence region of cellular environments. Efficient energy transfer of chemiexcitation energy to drive nanoparticle luminescence enables an attractive alternative luminescent signal enhancement mode to the cytotoxic polycationic polymeric enhancers that currently enable commercial dioxetane applications. This type of application could be used with the systems such as the Appplied Biosystems 1700 or the Applied Biosystems 8200 Cellular Detection System wherein the excitation lamp is turned off.

Chemiluminescent substrate-nanoparticle-bioanalyte conjugate assemblies can be used in a sensitive assay system for detection of an analyte captured on the nanoparticle surface by energy transfer (ET) from excited state dioxetane donor fragments to nanoparticle acceptors. According to one embodiment, the bioassay on the nanoparticle surface can comprise 1-50 or more molecules of a capture agent, (e.g., antibodies, oligonucleotides, receptors, lectins, and aptamers) conjugated to or ionically associated with the quantum dot surface.

In the case of an immunoassay, the capture of the analyte by the capture agent can be detected with a hydrolytic enzyme label attached to a detector antibody in a sandwich assay or attached to the captured analyte. When dioxetane fragmentation is initiated by hydrolytic enzyme activation, the potential energy of the four-membered peroxide ring is released to excite the aryl ester fragment to a singlet excited state. Relaxation of the singlet excited state fragment to ground state transfers energy to proximal nanoparticle acceptors, which then luminesce with emission governed by nanoparticle size.

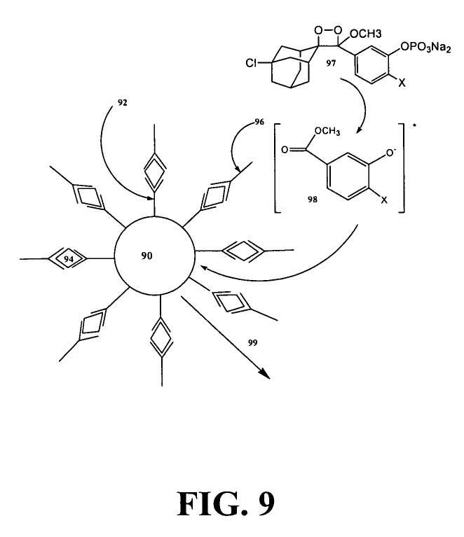

A sandwich type immunoassay using a chemiluminescent substrate/nanoparticle assembly is shown in FIG. 9. As shown in FIG. 9, a nanoparticle (90) comprising a plurality of covalently attached capture antibodies (92) is provided. Capture antibodies (92) can bind analyte (94) in the sample. Bound analyte (94) can then bind an enzyme labeled detection antibody (96). Enzyme activation of a dioxetane (97) added to the sample results in generation of chemiexcited species (98) that can transfer energy to the nanoparticle resulting in photoluminescent emission (99) from the nanoparticle. The photoluminescent emissions from the nanoparticle can have a wavelength of 480 nm to >700 nm.

The dioxetane thus operates as the source of excitation energy for generation of nanoparticle luminescence. The dioxetane-nanoparticle energy transfer assembly of this invention can use either electrostatic charge attractions to bind donor and acceptor energy transfer components together (i.e., ionic association) or covalent attachment of the dioxetane substrate to the nanoparticle surface (i.e., covalent association). The capture agent and dioxetane can be covalently attached using standard bioconjugation chemistries with a functionalized nanoparticle surface having, for example, amines, thiols, carboxylates, boronic acids, or alcohols as coupling sites. Ionic association can occur through interaction of negatively charged groups on the dioxetane and chemiexcited donor fragment with positively charged onium groups (e.g., ammonium, sulfonium, phosphonium) on the nanoparticle surface or through the converse design. Another example of bonding interaction occurs through interaction of ZnS surfaces with thiol or heteroarylamine groups on the dioxetane and chemiexcited donor fragment to form Zn—S or Zn—N bonds. These types of nanoparticle dioxetane detection assemblies and their advantages are described in detail above.

Assays using nanoparticle beads are also provided wherein the solid support/energy transfer acceptors are nanoparticle-embedded polymer beads comprising multiple molecules of a capture agent conjugated to the surface that can expose a population of nanoparticles on the bead surface to chemiexcited species capable of energy transfer to the nanoparticle.

The nanoparticle-supported bioassay design allows quantification of nanoparticles hosting multiplexed bioassays wherein discrete nanoparticles can represent discrete assays. Using nanoparticles as the assay support provides more rapid assay times due to radial diffusion, greater reproducibility through thousands to millions of replicates, and requires less sample and reagent (see, for example, Natan et al., “Nanoparticles for Bioanalysis”, Current Opinion in Chemical Biology 2003, 7:609-615). Chemiexcitation-initiated nanoparticle photoluminescence reduces noise, does not require an external excitation light source, and provides luminescent signal red-shifted beyond typical autofluorescence bandwidths associated with biological and cellular materials. The narrowed emission bandwidth of the nanoparticle acceptor enables easily resolvable luminescent signals in multichannel or multibead bioassay systems. Nanoparticle bioassays also allow for homogeneous assay formats where localized enzyme turnover generates localized chemiexcitation of the nanoparticle support correlated to nearby captured analyte detection.

The dioxetane-nanoparticle-bioanalyte conjugate assemblies can be used as sensitive bioassay nanoparticles for enzyme labeled bioassays in chip formats and solution assays. They can also be used for enzyme-linked or enzyme expression assays in cells. The capability of red-shifting the luminescent signal from autofluorescence bands and initiating the signal from chemiexcitation provides significantly lower background, increased signal to noise, and increased detection sensitivities.

Various exemplary embodiments of surface modified nanoparticles, nanoparticle/chemiluminescent substrate assemblies and of assays using these surface modified nanoparticle and nanoparticle assemblies are described below.

According to some embodiments, a nanoparticle is provided which comprises an inorganic semiconductor material which can produce photoluminescent emissions upon excitation via a non-radiative energy transfer process wherein a surface of the nanoparticle comprises cationic moieties. For example, the nanoparticle can comprise a cationic coating. Exemplary cationic coatings include, but are not limited to, polycationic polymers or co-polymers. The polycationic polymer or co-polymer can comprise ammonium, phosphonium or sulfonium moieties. The polycationic polymer or co-polymer can comprise quaternary onium moieties. Exemplary polycationic polymers or co-polymer include TBQ, TPQ, THQ and TOP. Alternatively, the coating can comprise a polyelectrolyte multilayer (PEM) coating that comprises alternating layers of polyanionic (e.g., poly(acrylate), poly(L-glutamate), poly(styrenesulfonate), or hyaluronate or combinations thereof) and polycationic polymers or co-polymers (e.g., TBQ, TPQ, THQ, BDMQ, TOP, poly(allylamine hydrochloride), poly(L-lysine), poly(ethyleneimine) or poly(L-arginine) and/or combinations thereof). The cationic moiety can be a dendron ligand. According to this embodient, the inorganic semiconductor material can comprise CdSe, CdS, CdTe, ZnS, ZnSe, ZnTe, MgTe, HgTe, Si or Si compounds and/or combinations thereof. For example, the inorganic semiconductor material can comprise CdSe and ZnS or CdTe and ZnS.

According to some embodiments, an assay is provided which comprises: combining a chemiluminescent compound, a nanoparticle comprising an inorganic semiconductor material and a sample, comprising an analyte, in aqueous solution, wherein the analyte comprises an enzyme; and wherein the chemiluminescent compound can be activated by the enzyme to produce an excited state donor, wherein the excited state donor associates with the nanoparticle such that the excited state donor transfers energy via a non-radiative process to the inorganic semiconductor material of the nano-particle which thereby produces photoluminescent emissions. The analyte can be the enzyme itself or the analyte can be labeled with the enzyme. The chemiluminescent compound can be a dioxetane, acridinium salt active ester, an acridan ester, an acridan thioester, an acridan enol phosphate, an enol phosphate, and luminol. Exemplary dioxetanes include dioxetanes represented by one of the following formulae 1-7: 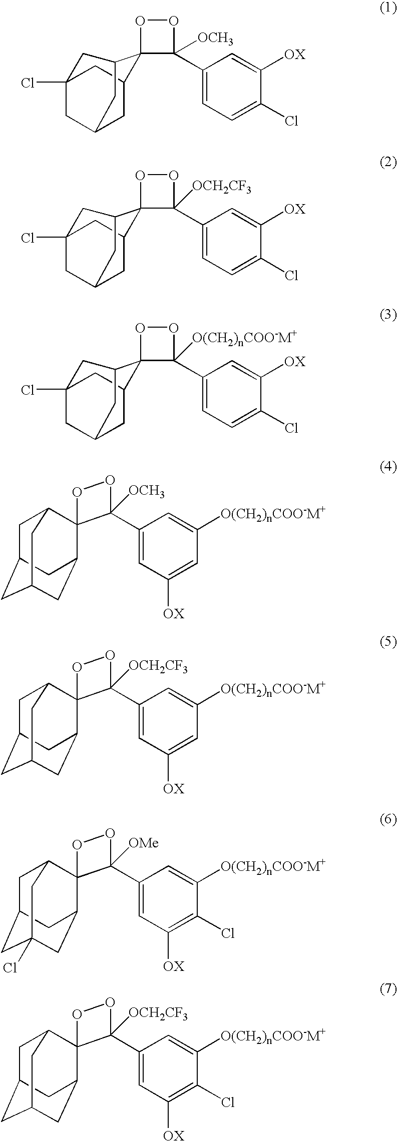
wherein n is an integer from 1-16, X is a phosphate or β-galactoside group and M⁺ is a Na⁺, K⁺, Li⁺, pyridinium, peralkylammonium or ammonium ion.

According to some embodiments, an enzyme substrate is provided which comprises: a nanoparticle comprising an inorganic semiconductor material; and at least one molecule of a chemiluminescent compound associated with the nanoparticle; wherein the chemiluminescent compound can be activated by an enzyme to produce an excited state donor; and wherein the excited state donor transfers energy via a non-radiative process to the inorganic semiconductor material which thereby produces photoluminescent emissions. In some embodiments, from 1 to 100,000 molecules of the chemiluminescent compound can be associated with the nanoparticle. In some embodiments, more than 100,000 molecules of a chemiluminescent compound can be associated with the nanoparticle. The chemiluminescent compound can be a dioxetane, an acridinium salt active ester, an acridan ester, an acridan thioester, an acridan enol phosphate, an enol phosphates or a luminol. The chemiluminescent compound can be associated with the nanoparticle via covalent bonding. The chemiluminescent compound can comprise a moiety represented by one of the following formulae 8-13: 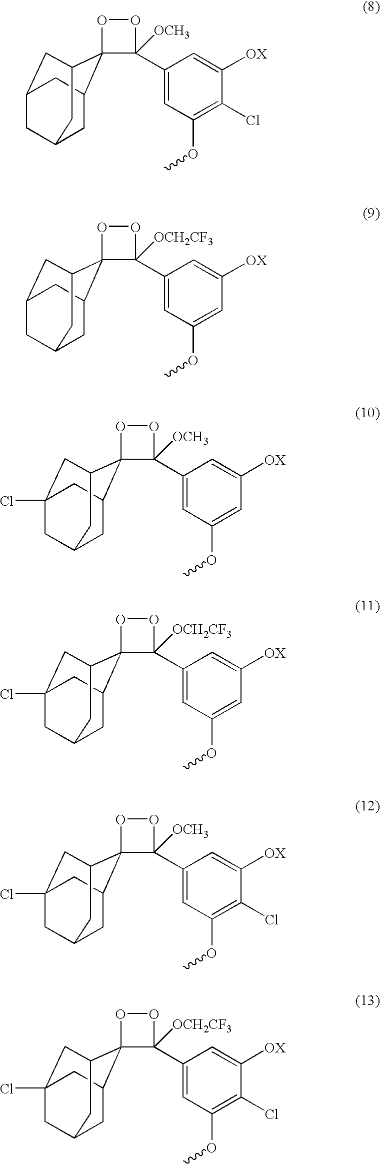
wherein X in each of the above formulae is a phosphate group or a β-galactoside group. The wavy bond in the above formulae represents either a covalent bond between the moiety and a surface of the nanoparticle or a linker group connecting the moiety to a surface of the nanoparticle. The inorganic semiconductor material can comprise CdSe, CdS, CdTe, ZnS, ZnSe, ZnTe, MgTe, HgTe, Si or Si compounds and/or combinations thereof. For example, the inorganic semiconductor can comprise CdSe and ZnS or CdTe and ZnS. The nanoparticle can be a bead comprising a plurality of nanoparticles of the inorganic semiconductor material embedded in a polymer or co-polymer.

The chemiluminescent compound of the enzyme substrate can also be associated with the nanoparticle via ionic bonding. The surface of the inorganic semiconductor material can comprise cationic moities such as quaternary onium moieties (e.g., quaternary ammonium, sulfonium, or phosphonium moieties). The nanoparticle can comprise a coating and the chemiluminescent compound(s) can be associated with the coating on the nanoparticle. The coating can comprise a polycationic polymer or co-polymer. The polycationic polymer or co-polymer can comprise quaternary onium moieties (e.g., quaternary ammonium, sulfonium, or phosphonium moieties). Exemplary polycationic polymers or co-polymers include, but are not limited to, TBQ, TPQ, THQ and TOP. The chemiluminescent compound can be a dioxetane represented by one of the following formulae 1-7: 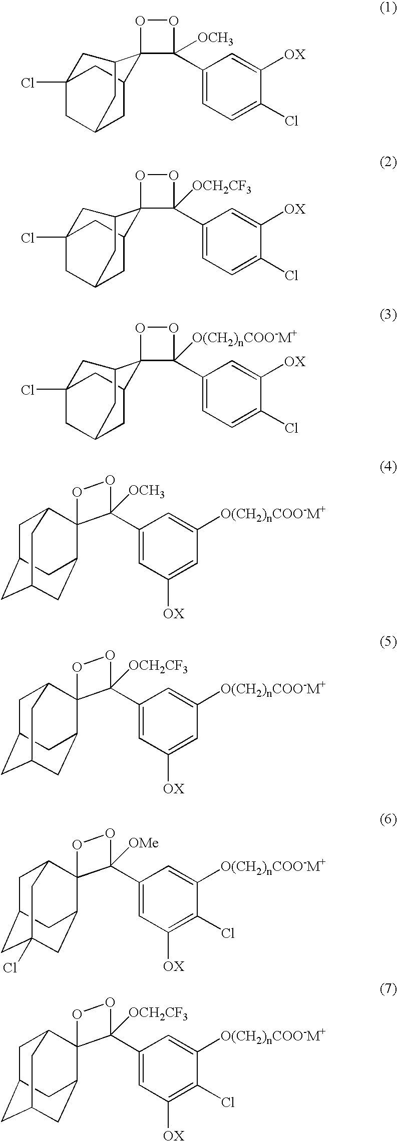
wherein: n is an integer from 1-16, X in the above formulae is a phosphate or β-galactoside group and M⁺ is a Na⁺, K⁺, Li⁺, pyridinium, peralkylammonium or or ammonium ion.

The chemiluminescent compound of the enzyme substrate can also be 10 associated with the nanoparticle via Zn—S or Zn—N bonding. The surface of the inorganic semiconductor material can comprise ZnS or ZnSe coatings that associate with chemiluminescent compounds having a thiol or heteroarylamine group. Bonding to the nanoparticle surface occurs by interaction of the surface with a group M on a linker that can be an alkylthio, arylthio, arylamino or heteroarylamino, having a displaceable proton in the pKₐ range of 5.0-9.0.

The chemiluminescent compound can be a dioxetane represented by one of the following formulae 14-19: 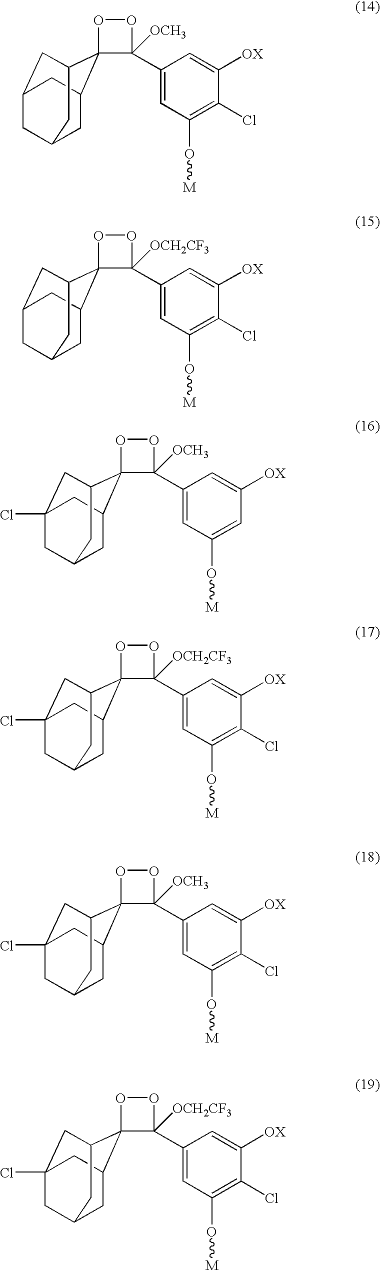
wherein X is phosphate or β-galactoside and wherein M is —SH or a heterocyclic amino group. Exemplary heterocyclic amino groups include the following:

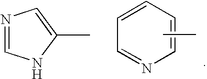

An enzyme substrate may further comprise one or more molecules of a capture agent associated with a surface of the nanoparticle. The one or more molecules of the capture agent can be associated with the surface of the nanoparticle via covalent or ionic bonding. In some embodiments, from 1 to 1,000 molecules of the capture agent can be associated with the surface of the nanoparticle. In some embodiments, more than 1,000 molecules of capture agent can be associated with the surface of the nanoparticle. The capture agent can be an antibody, a polynucleotide, an oligonucleotide, a polypeptide, a protein, a receptor, a lectin, or an aptamer.

According to some embodiments, an assay is provided which comprises: contacting an enzyme substrate as set forth in any of the above described embodiments of with a sample comprising an analyte, wherein the analyte comprises an enzyme capable of activating the chemiluminescent compound; allowing the analyte in the sample to activate the chemiluminescent compound; and detecting the photoluminescent emissions from the nanoparticle; wherein enzyme activation of the chemiluminescent compound results in generation of a product in its excited state which transfers energy in a non-radiative process to the nanoparticle which thereby produces photoluminescent emissions and wherein the photoluminescent emissions from the nanoparticle indicate the presence and/or amount of analyte in the sample. The analyte can be an enzyme capable of activating the chemiluminescent compound to generate an excited state donor or the analyte can be labeled with an enzyme capable of activating the chemiluminescent substrate to generate an excited state donor. The assay can be conducted in solution or on a solid support. The assay can also be a cellular assay.

According to some embodiments, an assay is also provided which comprises: contacting an enzyme substrate further comprising one or more molecules of a capture agent with the sample comprising an analyte; allowing analyte in the sample to associate with the capture agent; and detecting photoluminescent emissions from the nanoparticle; wherein the capture agent is capable of associating with the analyte, wherein enzyme activation of the chemiluminescent compound produces an excited state donor, wherein the excited state donor transfers energy in a non-radiative process to excite the inorganic semiconductor material to produce photoluminescent emissions and wherein the photoluminescent emissions from the nanoparticle indicate the presence and/or amount of analyte in the sample.

The analyte and the capture agent can both comprise polynucleotides wherein the capture agent is capable of hybridizing to the analyte. Alternatively, the analyte and the capture strand can both comprise polypeptides wherein the capture agent associates with the analyte via protein-protein interactions. The analyte can also comprise an antigen and the capture strand can be an antibody capable of binding the antigen wherein the capture agent associates with the analyte via antigen-antibody binding. Analyte in the sample can be labeled with an enzyme capable of activating the chemiluminescent substrate to generate an excited state donor. The assay can further comprise contacting the substrate with an enzyme labeled species capable of binding analyte associated with the capture agent, after allowing analyte in the sample to associate with the capture agent, wherein the enzyme label is capable of activating the chemiluminescent compound to generate an excited state donor. The assay can further comprise contacting the substrate with an enzyme labeled antibody capable of binding analyte associated with the capture agent after allowing analyte in the sample to associate with the capture agent, wherein the enzyme label is capable of activating the chemiluminescent compound to generate an excited state donor. The assay can be a specific binding pair assay. The analyte can comprise an enzyme capable of activating the chemiluminescent compound to generate an excited state donor. For example, the analyte can be labeled with the enzyme capable of activating the chemiluminescent compound to generate an excited state donor. The capture agent and analyte can be members of a specific binding pair selected from the group consisting of antigen:antibody, DNA:DNA conjugate, DNA:RNA conjugate, DNA:PNA conjugate, PNA:PNA conjugate, PNA-RNA conjugate, biotin-avidin conjugate, and protein-protein complex. The assay can be conducted in solution or on a solid support. The assay can also be a cellular assay. A cellular assay can be in vivo detection of an event inside a cell or it ca be in vitro detection of an agent in cellular components exposed by cell lysis.

According to some embodiments, an assay is provided which comprises: contacting a sample comprising a first analyte and a second analyte with a first substrate, the first substrate comprising a nanoparticle of an inorganic semiconductor material and at least one molecule of a first chemiluminescent compound associated with the nanoparticle, wherein the first chemiluminescent compound can be activated by a first enzyme to produce an excited state donor which transfers energy in a non-radiative process to the inorganic semiconductor material thereby producing a first photoluminescent emission and wherein the first analyte is capable of activating the first chemiluminescent compound. The assay further comprises contacting the sample with a second substrate, the second substrate comprising a nanoparticle of an inorganic semiconductor material and at least one molecule of a second chemiluminescent compound associated with the nanoparticle, wherein the second chemiluminescent compound can be activated by a second enzyme to produce an excited state donor which transfers energy in a non-radiative process to the nanoparticle thereby producing a second photoluminescent emission spectrally distinct from the first photoluminescent emission and wherein the second analyte is capable of activating the second chemiluminescent compound.

The assay can further comprise detecting the first and/or second photoluminescent emissions; wherein the first photoluminescent emission indicates the presence and/or amount of the first analyte in the sample, and wherein the second photoluminescent emission indicates the presence and/or amount of the second analyte in the sample. In the above described assay, the first analyte can be labeled with the first enzyme and the second analyte can be labeled with the second enzyme. Alternatively, the first analyte can be the first enzyme and the second analyte can be the second enzyme.

The assay according to these embodiments can be conducted in solution or on a solid support. The assay can be a cellular assay. The first and second chemiluminescent compounds can be different, and the nanoparticles to which the first and second chemiluminescent compounds are associated can have different characteristics (e.g., size) such that they each produce spectrally distinct photoluminescent emissions.

When the assay is conducted in solution, a chemiluminescent enhancer can be added to the solution. Exemplary chemiluminescent enhancers include, but are not limited to, polycationic polymers or co-polymers comprising quaternary onium moieties (e.g., quaternary ammonium, sulfonium, or phosphonium moieties). Exemplary polycationic polymers or co-polymers include, but are not limited to, TBQ, TPQ, THQ and TOP.

According to some embodiments, an assay for detecting the presence of a first and/or a second analyte in a sample is provided. The assay comprises: contacting the sample with a first substrate, the first substrate comprising a nanoparticle, at least one molecule of a first chemiluminescent compound associated with the nanoparticle, and at least one molecule of a first capture agent associated with the nanoparticle, wherein the first capture agent can associate with the first analyte, wherein the first chemiluminescent compound can be activated by a first enzyme to produce an excited state donor which transfers energy in a non-radiative process to the nanoparticle thereby producing a first photoluminescent emission. The assay further comprises contacting the sample with a second substrate, the second substrate comprising a nanoparticle, at least one molecule of a second chemiluminescent compound associated with the nanoparticle, and at least one molecule of a second capture agent associated with the nanoparticle, wherein the second capture agent can associate with the second analyte, wherein the second chemiluminescent compound can be activated by a second enzyme to produce an excited state donor which transfers energy in a non-radiative process to the nanoparticle thereby producing a second photoluminescent emission spectrally distinct from the first photoluminescent emission. The assay further comprises allowing the first analyte in the sample to associate with the first capture agent and allowing the second analyte in the sample to associate with the second capture agent and detecting the first and second photoluminescent emissions wherein the first photoluminescent emission indicates the presence and/or amount of the first analyte in the sample, and wherein the second photoluminescent emission indicates the presence and/or amount of the second analyte in the sample.

In the above described assay, the first and/or second analyte and the first and/or second capture agents can comprise polynucleotides, wherein the first capture agent is capable of hybridizing to the first analyte and wherein the second capture agent is capable of hybridizing to the second analyte. Alternatively, each of the first and second analytes and the first and second capture agents can comprise polypeptides, wherein the first capture agent can associate with the first analyte via protein-protein interactions and wherein the second capture agent can associate with the second analyte via protein-protein interactions. In addition, the first and second analyte can each comprise an antigen and the first and second capture strands each comprise an antibody capable of binding the first and second analytes, respectively, and wherein the first and second capture agents associate with the first and second analytes via antigen-antibody binding. The first analyte can be labeled with the first enzyme and the second analyte is labeled with the second enzyme. The first capture agent and the first analyte and the second capture agent and the second analyte can be members of a specific binding pair. For example, the first and second capture agents and the first and second analytes can each be members of a specific binding pair. Exemplary binding pairs include: antigen:antibody complexes, DNA:DNA complexes, DNA:RNA complexes, DNA:PNA complexes, PNA:PNA complexes, PNA-RNA complexes, biotin-avidin complexes, and protein-protein complex.

The assay described above can further comprise: contacting the first substrate with a first species capable of binding the first analyte associated with the first capture agent, after allowing first analyte in the sample to associate with the first capture agent, wherein the first species is labeled with the first enzyme; and contacting the second substrate with a second species capable of binding the second analyte associated with the second capture agent after allowing second analyte in the sample to associate with the second capture agent, wherein the second species is labeled with the second enzyme. The first and second species can be antibodies capable of binding the first and second analyte, respectively.

The assay according to these embodiments can be conducted in solution or on a solid support. The assay can also be a cellular assay. The first and/or second photoluminescent emissions can be detected simultaneously or sequentially. The sample can also be contacted with the first and second substrates simultaneously or sequentially. The first and second chemiluminescent compounds as well as the first and second enzymes can be the same and the nanoparticles to which the first and second chemiluminescent compounds are associated can have different characteristics (e.g., sizes) such that they each produce spectrally distinct photoluminescent emissions.

### EXPERIMENTAL

Synthesis of Aqueous-Soluble Carboxylate-coated CdSe/ZnS Nanoparticles

CdSe/ZnS nanoparticles (8-20 mg) were suspended in 0.1-0.2 ml mercaptoacetic acid and heated to 130° C. for 3 minutes. The resulting mixture was cooled to room temperature, triturated with dichloromethane (5×3 ml), and the orange solid was air-dried. The modified nanoparticles were dissolved in 0.2M NaOH at a concentration of up to 30 mg/ml, and diluted with 0.1 M AMP (2-amino-2-methyl-1-propanol) buffer, pH 9.5, or diluted with water, to the desired concentration.

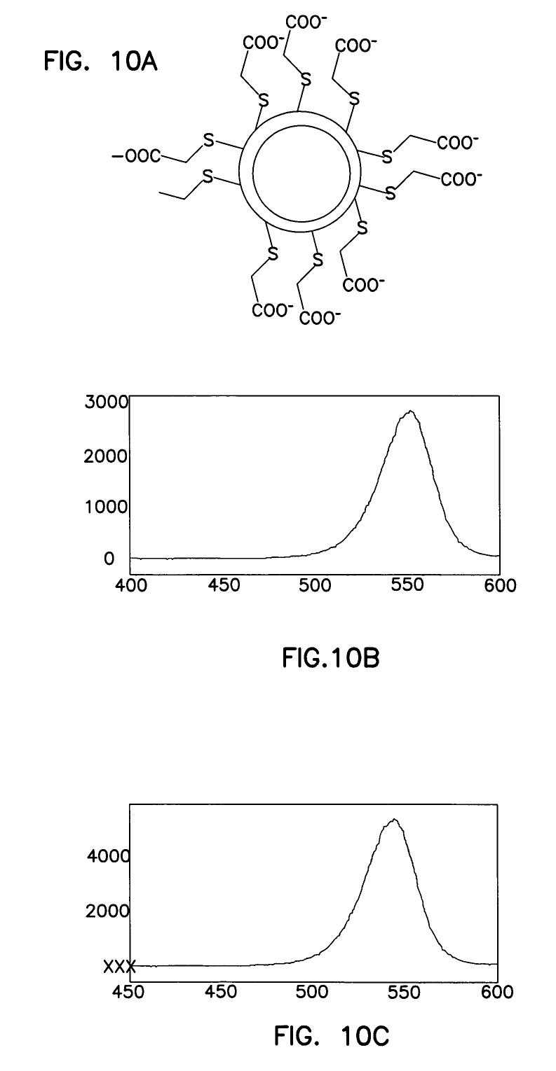

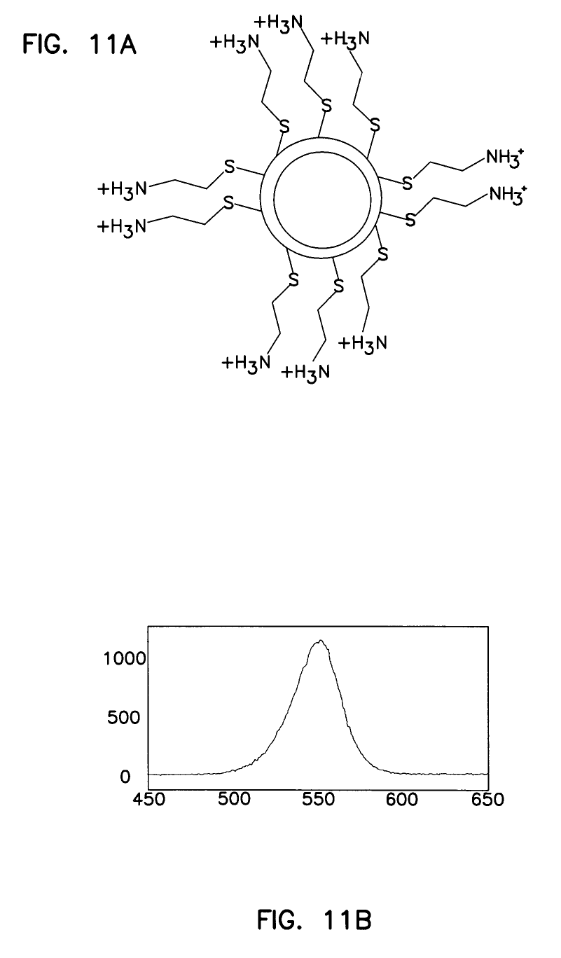

FIG. 10A is a schematic illustrating the structure of the carboxylate-coated CdSe/ZnS nanoparticles. FIGS. 10B and 10C are graphs showing emission intensity as a function of wavelength for fresh (FIG. 10A) and aged (i.e., four month old) (FIG. 10B) solutions of the carboxylate-coated CdSe/ZnS nanoparticles in 0.1 M AMP buffer at a pH of 9.5.

Synthesis of Aqueous-soluble Ammonium-coated CdSe/ZnS Nanoparticles

CdSe/ZnS nanoparticles (9 mg) were mixed with solid 2-aminoethanethiol hydrochloride and heated to ˜100° C. until the solids dissolved in a melt. After the resulting melt was cooled to room temperature, the solid was dissolved in water (2 ml), and filtered through a small paper plug to remove white solid. The orange solution was then filtered through a Centricon YM-30 filter (30 kD MWCO) via centrifugation, leaving a viscous orange nanoparticle solution on the filter. The orange nanoparticle “gel” was dissolved in water and washed (3×2 ml) to remove excess mercaptoacetic acid; the filtrates were clear and colorless indicating that the modified nanoparticles remained on the filter membrane. The washed nanoparticles were then dissolved in water to the desired concentration, and the solution was recovered from the filter membrane by pipette.

FIG. 11A is a schematic illustrating the structure of the ammonium-coated CdSe/ZnS nanoparticles. FIG. 11B is a graph showing the intensity of the nanoparticle emissions as a function of wavelength.

Energy Transfer from CDP-Star® to Carboxylate-Coated CdSe/ZnS Nanoparticles

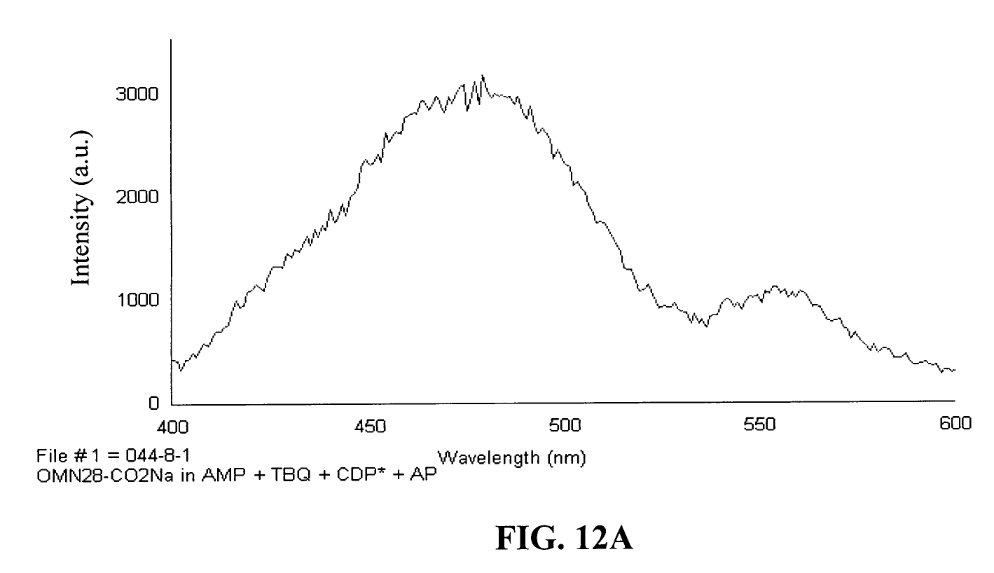

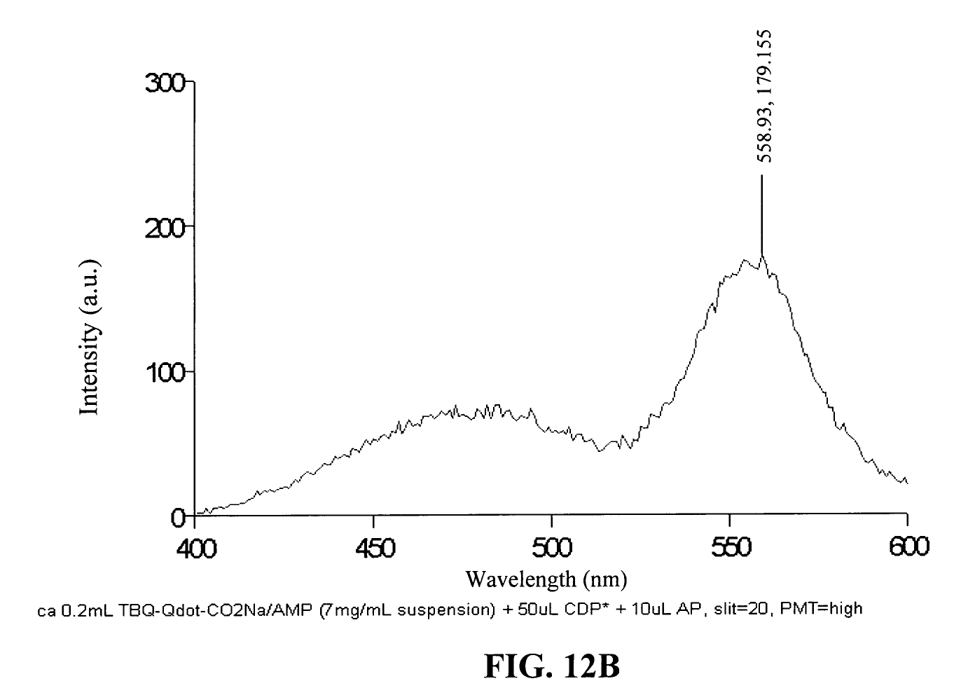

A. Simple Mixing: Carboxylate-coated nanoparticles (1 mg) were dissolved in 0.2M NaOH (0.2 ml), and the solution was diluted with 0.1M AMP buffer, pH 9.5 (0.8 ml). Sapphire II solution (10 mg/ml; 0.2 ml) and CDP-Star® (12.5 mg/ml; 50 ml) were added to the carboxylate nanoparticles. Alkaline phosphatase (Biozyme Laboratories #ALP112G, diluted 1:1000, 10 ml) was added to initiate CDP-Star® decomposition, and the resulting luminescence emission was collected on a Spex Fluorolog Spectrometer in the chemiluminescent mode. Chemiluminescent emission data are depicted in FIG. 12A which is a graph of emissions intensity as a function of wavelength. As can be seen from FIG. 12A, the data showed mostly CDP-Starφ emission with a maximum at 470 nm, with some observed carboxylate-nanoparticle emission at a maximum at 559 nm. The red-shifted nanoparticle emission is attributed to excitation of the carboxylate-nanoparticles by energy transfer from excited state CDP-Star® decomposition fragments.

B. TBQ-treated Carboxylate-nanoparticles: Carboxylate-coated nanoparticles (8 mg) were dissolved in 0.2M NaOH (0.1 ml), and the solution was diluted with 0.1 M AMP buffer, pH 9.5 (0.5 ml). To this solution, Sapphire II solution (10 mg/ml; 0.2 ml) was added; a red solid immediately precipitated. The reaction mixture was diluted-with water to 3 ml total volume, centrifuged, and dried by air and under vacuum to a Sapphire treated carboxylate-nanoparticle solid (7 mg). The nanoparticle solid (7 mg) was sonicated in 0.1M AMP buffer, pH 9.5 (1 ml) for 1 hr to form a suspension. A portion of the suspension (0.2 ml) was placed in a well; CDP-Star® (12.5 mg/ml; 50 ml) and alkaline phosphatase (Biozyme Laboratories #ALP112G, diluted 1:1000, 10 ml) were added to initiate CDP-Star® decomposition. The resulting luminescence emission was collected on a Cary Eclipse Spectrometer in the chemiluminescence mode. The chemiluminescent emission data is depicted in FIG. 12B which is a graph of emission intensity as a function of wavelength. As can be seen from FIG. 12B, the data showed increased carboxylate-nanoparticle emission with a maximum at 559 nm, with CDP-Star® emission also evident at a 470 nm maximum wavelength. The red-shifted nanoparticle emission is attributed to excitation of the carboxylate-nanoparticles by energy transfer from excited state CDP-Star® decomposition fragments.

Energy Transfer from Dioxetane-N⁺(Me)₃ to Carboxylate Coated CdSe/ZnS Nanoparticles

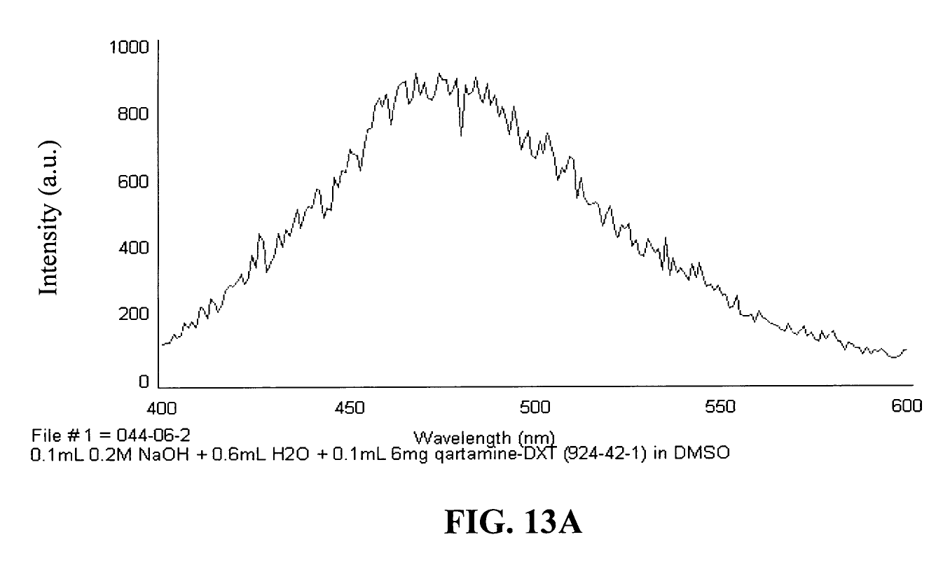

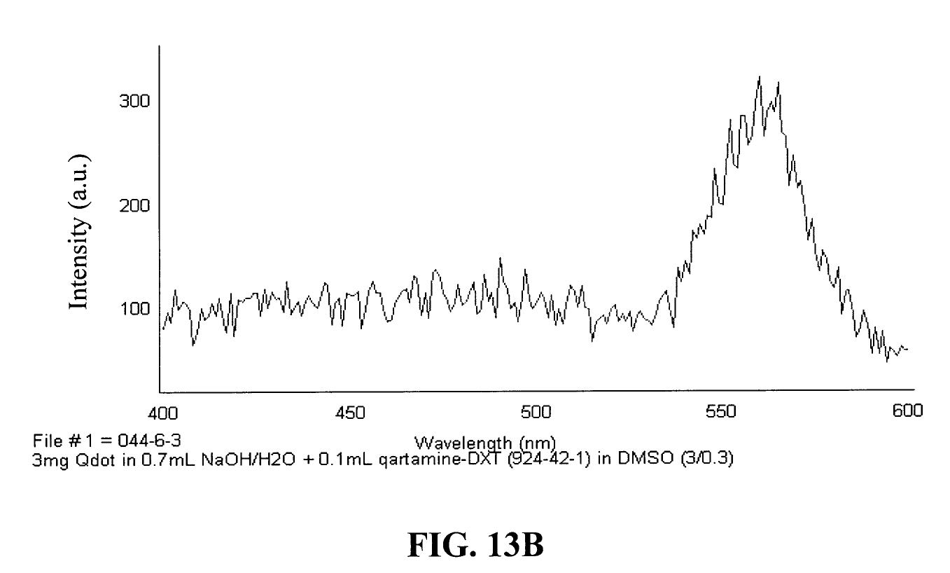

Carboxylate coated CdSe/ZnS nanoparticles (3 mg) were dissolved in 0.2M NaOH (0.1 ml) and diluted with water (0.6 ml). To this solution was added a solution of a trimethylammonium dioxetane (20 mg/ml DMSO, 0.1 ml). Excess NaOH initiated trimethylammonium dioxetane decomposition, and the resulting luminescence emission data was collected on a Spex Fluorolog Spectrometer in the chemiluminescent mode. This chemiluminescent emission data is depicted graphically in FIG. 13B. FIG. 13B is a graph showing emission intensity as a function of wavelength for triethylammonium dioxetane in the presence of carboxylate nanoparticles showing an emission maximum of 559 nm. As can be seen from FIG. 13B, the data showed carboxylate-nanoparticle emission with a maximum at 559 nm. The red-shifted nanoparticle emission is attributed to excitation of the carboxylate-nanoparticle by energy transfer from excited state trimethylammonium dioxetane decomposition fragments. In the absence of the carboxylate coated nanoparticles, trimethylammonium dioxetane emission, produced by NaOH initiated dioxetane decomposition, exhibited a maximum at ˜470 nm. FIG. 13A is a graph showing emission intensity as a function of wavelength for triethylammonium dioxetane in the absence of the carboxylate nanoparticles showing an emission maximum at approximately 470 nm.

The section headings used in this application are for organizational purposes only and are not to be construed as limiting the subject matter described herein in any way.

While the foregoing specification teaches the principles of the present invention, with examples provided for the purpose of illustration, it will be appreciated by one skilled in the art from reading this disclosure that various changes in form and detail can be made without departing from the true scope of the invention.

1. A nanoparticle comprising an inorganic semiconductor material which can produce photoluminescent emissions upon excitation via a non-radiative energy transfer process wherein a surface of the nanoparticle comprises cationic moieties.

2. The nanoparticle of claim 1, wherein the nanoparticle comprises a cationic coating.

3. The nanoparticle of claim 2, wherein the cationic coating comprises a polycationic polymer or co-polymer.

4. The nanoparticle of claim 3, wherein the polycationic polymer or co-polymer comprises ammonium, phosphonium or sulfonium moieties.

5. The nanoparticle of claim 3, wherein the polycationic polymer or co-polymer comprises quaternary onium moieties.

6. The nanoparticle of claim 3, wherein the polycationic polymer or co-polymer is selected from the group consisting of TBQ, TPQ, THQ and TOP.

7. The nanoparticle of claim 2, wherein the coating comprises a polyelectrolyte multilayer (PEM) coating comprising alternating layers of polyanionic and polycationic polymers or co-polymers.

8. The nanoparticle of claim 7, wherein the polyanionic polymers or copolymers are selected from the group consisting of poly(acrylate), poly(L-glutamate), poly(styrenesulfonate), hyaluronate and combinations thereof.

9. The nanoparticle of claim 7, wherein the polycationic polymers or copolymers are selected from the group consisting of TBQ, TPQ, THQ, BDMQ, TOP, poly(allylamine hydrochloride), poly(L-lysine), poly(ethyleneimine), poly(L-arginine) and combinations thereof.

10. The nanoparticle of claim 1, wherein the cationic moiety is a dendron ligand.

11. The nanoparticle of claim 1, wherein the inorganic semiconductor material comprises CdSe, CdS, CdTe, ZnS, ZnSe, ZnTe, MgTe, HgTe, Si, Si compounds or combinations thereof.

12. The nanoparticle of claim 1, wherein the inorganic semiconductor material comprises CdSe and ZnS or CdTe and ZnS.

13. An assay comprising:
combining a chemiluminescent compound, a nanoparticle comprising an inorganic semiconductor material and a sample comprising an analyte in aqueous solution;
wherein the analyte comprises an enzyme; and
wherein the chemiluminescent compound can be activated by the enzyme to produce an excited state donor, wherein the excited state donor associates with the nanoparticle such that the excited state donor transfers energy via a non-radiative process to the inorganic semiconductor material of the nano-particle which thereby produces photoluminescent emissions.

14. The assay of claim 13, wherein the analyte is the enzyme.

15. The assay of claim 13, wherein the analyte is labeled with the enzyme.

16-18. (canceled)

19. An enzyme substrate comprising:
a nanoparticle comprising an inorganic semiconductor material; and
at least one molecule of a chemiluminescent compound associated with the nanoparticle;
wherein the chemiluminescent compound can be activated by an enzyme to produce an excited state donor; and
wherein the excited state donor transfers energy via a non-radiative process to the inorganic semiconductor material which thereby produces photoluminescent emissions.

20-43. (canceled)

44. The substrate of claim 19, further comprising one or more molecules of a capture agent associated with a surface of the nanoparticle.

45-48. (canceled)

49. An assay comprising:
contacting an enzyme substrate as set forth in claim 19 with a sample comprising an analyte, wherein the analyte comprises an enzyme capable of activating the chemiluminescent compound;
allowing the analyte in the sample to activate the chemiluminescent compound; and
detecting photoluminescent emissions from the nanoparticle;
wherein enzyme activation of the chemiluminescent compound results in generation of a product in its excited state which transfers energy in a non-radiative process to the nanoparticle which thereby produces photoluminescent emissions and wherein the photoluminescent emissions from the nanoparticle indicate the presence and/or amount of analyte in the sample.

50-52. (canceled)

53. An assay comprising:
contacting an enzyme substrate as set forth in claim 44 with a sample comprising an analyte;
allowing analyte in the sample to associate with the capture agent; and
detecting photoluminescent emissions from the nanoparticle;
wherein the capture agent is capable of associating with the analyte, wherein enzyme activation of the chemiluminescent compound produces an excited state donor, wherein the excited state donor transfers energy in a non-radiative process to excite the inorganic semiconductor material to produce photoluminescent emissions and wherein photoluminescent emissions from the nanoparticle indicate the presence and/or amount of analyte in the sample.

54-64. (canceled)

65. An assay comprising:
contacting a sample comprising a first analyte and a second analyte with a first substrate, the first substrate comprising a nanoparticle of an inorganic semiconductor material and at least one molecule of a first chemiluminescent compound associated with the nanoparticle, wherein the first chemiluminescent compound can be activated by a first enzyme to produce an excited state donor which transfers energy in a non-radiative process to the inorganic semiconductor material thereby producing a first photoluminescent emission and wherein the first analyte is capable of activating the first chemiluminescent compound;
contacting the sample with a second substrate, the second substrate comprising a nanoparticle of an inorganic semiconductor material and at least one molecule of a second chemiluminescent compound associated with the nanoparticle, wherein the second chemiluminescent compound can be activated by a second enzyme to produce an excited state donor which transfers energy in a non-radiative process to the nanoparticle thereby producing a second photoluminescent emission spectrally distinct from the first photoluminescent emission and wherein the second analyte is capable of activating the second chemiluminescent compound;
detecting the first and second photoluminescent emissions;
wherein the first photoluminescent emission indicates the presence and/or amount of the first analyte in the sample, and wherein the second photoluminescent emission indicates the presence and/or amount of the second analyte in the sample.

66-75. (canceled)

76. An assay for detecting the presence of a first and a second analyte in a sample comprising:
contacting the sample with a first substrate, the first substrate comprising a nanoparticle, at least one molecule of a first chemiluminescent compound associated with the nanoparticle, and at least one molecule of a first capture agent associated with the nanoparticle, wherein the first capture agent can associate with the first analyte, wherein the first chemiluminescent compound can be activated by a first enzyme to produce an excited state donor which transfers energy in a non-radiative process to the nanoparticle thereby producing a first photoluminescent emission;
contacting the sample with a second substrate, the second substrate comprising a nanoparticle, at least one molecule of a second chemiluminescent compound associated with the nanoparticle, and at least one molecule of a second capture agent associated with the nanoparticle, wherein the second capture agent can associate with the second analyte, wherein the second chemiluminescent compound can be activated by a second enzyme to produce an excited state donor which transfers energy in a non-radiative process to the nanoparticle thereby producing a second photoluminescent emission spectrally distinct from the first photoluminescent emission;
allowing the first analyte in the sample to associate with the first capture agent and allowing the second analyte in the sample to associate with the second capture agent; and
detecting the first and second photoluminescent emissions;
wherein the first photoluminescent emission indicates the presence and/or amount of the first analyte in the sample, and wherein the second photoluminescent emission indicates the presence and/or amount of the second analyte in the sample.

77-82. (canceled)

83. The assay of claim 76, further comprising:
contacting the first substrate with a first species capable of binding the first analyte associated with the first capture agent, after allowing first analyte in the sample to associate with the first capture agent, wherein the first species is labeled with the first enzyme; and
contacting the second substrate with a second species capable of binding the second analyte associated with the second capture agent after allowing second analyte in the sample to associate with the second capture agent, wherein the second species is labeled with the second enzyme.

84-88. (canceled)

89. The assay of claim 76, wherein the first and second chemiluminescent compounds and the first and second enzymes are the same and wherein the nanoparticles to which the first and second chemiluminescent compounds are associated produce spectrally distinct photoluminescent emissions.
